(19) United States
(12) Patent Application Publication
(10) Pub. No.: US 2008/0269222 A1
(43) Pub. Date: Oct. 30, 2008

(54) Use of Delmopinol in the Treatment of Acne

(76) Inventor: Jonathan Douglas Lane, Surrey (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/993,914

(22) PCT Filed: Jul. 24, 2006
(86) PCT No.: PCT/GB2006/002770

§ 371 Date: Jul. 14, 2008

(30) Foreign Application Priority Data
Jul. 22, 2005 (GB) 051538.6

(51) Int. Cl. A61K 31/5375; A61Q 19/00; A61P 17/10

(52) U.S. Cl. 514/238.8; 514/231.2

(57) ABSTRACT

The present invention relates to the use of a morpholino compound having the general formula: (I) wherein R₁ is a straight or branched alkyl group containing 8 to 16 carbon atoms at the 2- or 3-position of the morpholino ring, and R₂ is a straight or branched alkyl group containing 2 to 10 carbon atoms, substituted with a hydroxy group except in the alpha-position, or pharmaceutically acceptable salts thereof, in the manufacture of a composition for the treatment of acne.

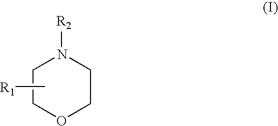

### FIELD OF THE INVENTION

The present invention relates to the treatment of acne, cosmetic methods of improving the appearance of skin and compositions for use in such methods.

### BACKGROUND TO THE INVENTION

Acne is the general term used to refer to a number of skin disorders characterised by irritation of pilosebaceous units in the skin (Krautheim and Gollnick, Clinics in Dermatology, 2004; 22:398-407).

Acne is a common skin complaint that affects many people at some point in their lives. Acne occurs at hair follicles, which are cavities in the surface of the skin containing hairs that extend from the deep layers of the skin to protrude through pores at the surface. A sebaceous gland secretes sebum into the hair follicle, which drains to the skin surface to provide lubrication and prevent the skin from drying out.

Acne occurs when the pore is blocked (for example by dead skin cells or through excessive production of sebum) so that, instead of draining onto the skin surface, the sebum collects in the follicle. The blocked follicle may then become infected with bacteria. Propionibacteria, that are normally found on the skin, are associated with the progression of acne, in particular Propionibacterium acnes (P. acnes).

Various types of acne are known and the most common form is Acne Vulgaris. In a mild form of Acne Vulgaris, the pore may expose a sebum plug, the surface of which turns black. This is known as a “blackhead” or “open comedone”. In other mild cases, the pore remains closed to the external environment and the trapped sebum and bacteria remain just below the surface of the skin. This is known as a “whitehead” or “closed comedone”. Whiteheads and blackheads are relatively minor non-inflammatory complaints and are primarily considered to be a cosmetic rather than a medical problem.

However, acne becomes more serious when the blocked follicle becomes inflamed leading to a swollen, reddened appearance from the surface, forming papules and pustules. It is thought that inflammation is induced mainly by an immunologic reaction to extracellular products of P. acnes that colonise the blocked pore (Zouboulis, Clinics in Dermatology, 2004; 22:360-366). The inflamed follicle can rupture onto the skin forming pustule heads. In serious cases, an intensely inflamed follicle can rupture under the skin, forming nodules and cysts, leading to deep scarring. Acne conglobata is the term given to the most severe form of acne.

Acne is unsightly and can cause severe scarring, in particular on the face. In addition to these physical effects, acne can cause severe psychological effects such as low self-esteem and depression.

Many topical treatments are currently used to treat acne. These typically contain active ingredients that have antimicrobial properties. Benzoyl peroxide is an important example of such an antimicrobial agent. Antibiotics are also commonly used, both topically and systemically, which has resulted in the development of bacterial resistance (as discussed in “European Surveillance Study on the Antibiotic Susceptibility of Propionibacterium Acnes”, C. Oprica and C. E. Nord; Clinical Microbiology and Infection, Volume 11. No. 3, March 2005, pages 204 to 213).

Although existing acne treatments are effective to some degree, there is room for improvement in this field and there remains a strong need for effective acne treatments.

### SUMMARY OF THE INVENTION

The present invention is based on the surprising discovery that delmopinol, and its derivatives, is useful in the treatment of acne.

According to a first aspect, the present invention relates to use of a morpholino compound having the general formula

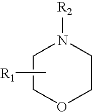

wherein R₁ is a straight or branched alkyl group containing 8 to 16 carbon atoms at the 2- or 3-position of the morpholino ring, and R₂ is a straight or branched alkyl group containing 2 to 10 carbon atoms, substituted with a hydroxy group except in the alpha-position, or pharmaceutically acceptable salts thereof, in the manufacture of a medicament for the treatment of acne.

According to a second aspect, the present invention relates to a cosmetic method of improving the appearance of skin, the method comprising the step of applying a composition to the skin, characterised in that the composition comprises a morpholino compound having the general formula

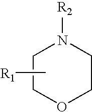

wherein R₁ is a straight or branched alkyl group containing 8 to 16 carbon atoms at the 2- or 3-position of the morpholino ring, and R₂ is a straight or branched alkyl group containing 2 to 10 carbon atoms, substituted with a hydroxy group except in the alpha-position, or pharmaceutically acceptable salts thereof.

According to a third aspect, the present invention relates to a composition suitable for dermatological application characterised in that it comprises a morpholino compound having the general formula

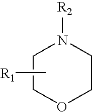

wherein R₁ is a straight or branched alkyl group containing 8 to 16 carbon atoms at the 2- or 3-position of the morpholino ring, and R₂ is a straight or branched alkyl group containing 2 to 10 carbon atoms, substituted with a hydroxy group except in the alpha-position, or pharmaceutically acceptable salts thereof.

According to a fourth aspect, the present invention provides a method of treating acne, the method comprising topical application to the skin of a composition comprising a morpholino compound having the general formula

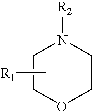

wherein R₁ is a straight or branched alkyl group containing 8 to 16 carbon atoms at the 2- or 3-position of the morpholino ring, and R₂ is a straight or branched alkyl group containing 2 to 10 carbon atoms, substituted with a hydroxy group except in the alpha-position, or pharmaceutically acceptable salts thereof.

### DESCRIPTION OF THE INVENTION

The claimed morpholino compounds are beneficial in the treatment of acne.

As used herein, the term “acne” refers to any skin condition comprising a blocked pore of a pilosebaceous unit. The term “acne” includes the presence of white heads (closed comedones), blackheads (open comedones), papules, pustules, nodules and cysts. The term “acne” includes non-inflammatory acne, such as minor blackheads and whiteheads, and inflammatory acne wherein an immune response causes the blocked follicle to become inflamed, causing pustules, nodules or cysts. Preferably, the acne is characterised by the presence of Propionibacterium acnes.

In a preferred embodiment, the acne is Acne vulgaris. All forms of Acne vulgaris, from mild through moderate to severe, are within the scope of the invention. The severe forms of Acne vulgaris are sometimes referred to as Acne congloblata and Acne fulminans. Acne congloblata is the most severe form of acne vulgaris and is characterised by numerous lesions which are often connected. Acne fulminans is an acute onset version of Acne congloblata. The term “acne” also includes folliculitis, in particular gram-negative folliculitis, and pyoderma faciale, a type of facial acne that affects only females.

The claimed morpholino compounds are known per se as disclosed in U.S. Pat. No. 4,894,221 and U.S. Pat. No. 5,082,653, which are incorporated herein by reference.

The first aspect of the present invention relates to the use of the claimed compounds in the treatment of acne.

In the present invention, the morpholino compounds are defined according to the general formula shown above. In a preferred embodiment of the present invention, the sum of carbon atoms in the groups R₁ and R₂ is at least 10, and is preferably between 10 and 20. In a further preferred embodiment, the R₂ group terminates with the hydroxy group.

The preferred morpholino compound for use in the present invention is 3-(4-propyl-heptyl)-4-(2-hydroxyethyl) morpholine which is commonly known as Delmopinol (CAS No. 79874-76-3).

The morpholino compounds of the present invention can be used in their free base form or as a pharmaceutically acceptable salt thereof. Some examples of pharmaceutically acceptable salts are the salts of acids such as acetic acid, phosphoric, acid, boric acid, hydrochloric acid, maleic acid, benzoic acid, citric acid, malic acid, oxalic acid, tartaric acid, succinic acid, glutaric acid, gentisic acid, valeric acid, gallic acid, beta-resorcyclic acid, acetyl salicylic acid, salicylic acid, perchloric acid, barbituric acid, sulfanilic acid, phytic acid, p-nitro benzoic acid, stearic acid, palmitic acid, oleic acid, myristic acid, lauric acid and the like. The most preferred salt form is of hydrochloric acid. A preferred compound is delmopinol hydrochloride (CAS No. 98092-92-3).

The claimed compounds can be manufactured by any known method, for example, that disclosed in U.S. Pat. No. 5,082,653 and WO 90/14342.

The claimed morpholino compounds are not thought to have any substantial antimicrobial action themselves. Therefore, the claimed compounds may advantageously be used in combination with a treatment that reduces the number of bacteria. For example, a composition containing the claimed morpholino compounds can be used as part of a skin cleansing routine.

A claimed morpholino compound is preferably used in combination with an antimicrobial agent. The antimicrobial agent can be any pharmaceutically acceptable compound which is effective against the bacteria associated with acne, for example, benzoyl peroxide.

Topical retinoids, which are thought to have an anti-inflammatory and indirect antibacterial effect, can also be used in combination with a claimed morpholino compound. Suitable retinoids will be apparent to the skilled person, and include tretinoin, isotretinoin, adapalene and tazortene.

An antibiotic may be used in a composition comprising a claimed morpholino compound. Any antibiotic that is effective against the bacteria associated with acne may be used. Preferably, antibiotics are selected from the group consisting of clindamycin, erythromycin, benzylpencillin, tetracycline, chloramphenicol, vancomycin and linezolid.

An anti-inflammatory agent may be used in a composition comprising a claimed morpholino compound. The skilled person will realise that the use of an anti-inflammatory agent is particularly useful in the treatment of inflammatory acne. Steroidal anti-inflammatories such as cortisone, or non-steroidal anti-inflammatories (NSAIDS), such as aspirin and ibuprofen, that inhibit cyclooxygenase isoenzymes are within the scope of the invention.

For the avoidance of doubt, all combinations of each of the composition ingredients described herein are within the scope of the invention.

The claimed morpholino compounds can also be used to have the cosmetic effect of improving the appearance of the skin in accordance with the second aspect of the present invention. The claimed compounds improve the appearance of skin not only by treating unsightly acne but also by discouraging the formation of acne.

The compositions for use in the present invention should be suitable for topical application to the skin and consist of the claimed compound in a pharmaceutically acceptable cream, ointment or gel. The composition can be hydrophillic or hydrophobic. The composition can be an aqueous composition, although other suitable solvents, such as alcohols or other organic solvents, may be used. A combination of solvents may also be used.

A suitable cream may be prepared by incorporating the active compound in a topical vehicle such as light liquid paraffin, dispersed in a aqueous medium using surfactants. An ointment may be prepared by mixing the active compound with a topical vehicle such as a mineral oil or wax. A gel may be prepared by mixing the active compound with a topical vehicle comprising a gelling agent.

Topically administrable compositions may also comprise a matrix in which a pharmaceutically active compound of the present invention is dispersed so that the compound is held in contact with the skin in order to administer the compounds transdermally.

The claimed morpholino compound should be included in the composition at a level at which it is effective which can be determined on the basis of the severity of the condition. The compound of the invention may be present in any suitable concentration. Typically, the compound is present in a concentration of from 0.01% (w/v) to 20% (w/v) e.g. 15% (w/v), preferably from 0.01% (w/v) to 10% (w/v), preferably from 0.1% (w/v) to 5% (w/v) and most preferably from 1% (w/v) to 3% (w/v) e.g. 2% (w/v). In general, a higher level of morpholino compound will be suitable for use in the medical treatment of acne than for the cosmetic use of improving the appearance of the skin. A suitable level can easily be selected by a person skilled in the art.

Where an antimicrobial, topical retinoid, antibiotic, anti-inflammatory agent and/or other additional component is included in the composition, these are also included at an effective level which can easily be determined by a person skilled in the art.

1. A method for the treatment of acne wherein said method comprises administering, to a patient in need of such treatment, a morpholino compound having the general formula 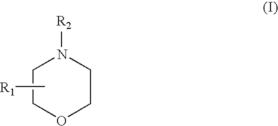
wherein R₁ is a straight or branched alkyl group containing 8 to 16 carbon atoms at the 2- or 3-position of the morpholino ring, and R₂ is a straight or branched alkyl group containing 2 to 10 carbon atoms, substituted with a hydroxy group except in the alpha-position, or pharmaceutically acceptable salts thereof, in the manufacture of a composition for the treatment of acne.

2. The method, according to claim 1 wherein the sum of the carbon atoms in the groups R₁ and R₂ is at least 10.

3. The method, according to claim 1 wherein R₂ terminates with a hydroxy group.

4. The method, according to claim 1, wherein the morpholino compound is 3-(4-propyl-heptyl)-4-(2-hydroxyethyl) morpholine.

5. The method, according to claim 1, which further comprises administering an antimicrobial agent to the patient.

6. The method, according to claim 5, wherein the antimicrobial agent is an antibiotic selected from the group consisting of clindamycin, erythromycin, benzylpencillin, tetracycline, chloramphenicol, vancomycin and linezolid.

7. The method, according to claim 1, wherein said method further comprises administering an anti-inflammatory agent to the patient.

8. A cosmetic method of improving the appearance of the skin of a patient, the method comprising the step of applying a composition to the skin wherein the composition comprises a morpholino compound having the general formula 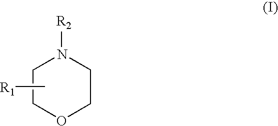
wherein R₁ is a straight or branched alkyl group containing 8 to 16 carbon atoms at the 2- or 3-position of the morpholino ring, and R₂ is a straight or branched alkyl group containing 2 to 10 carbon atoms, substituted with a hydroxy group except in the alpha-position, or pharmaceutically acceptable salts thereof.

9. The method according to claim 1, wherein said method comprises topically applying, to the skin of a patient, a composition comprising a morpholino compound having the general formula 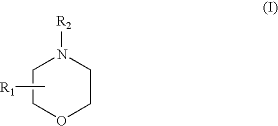
wherein R₁ is a straight or branched alkyl group containing 8 to 16 carbon atoms at the 2- or 3-position of the morpholino ring, and R₂ is a straight or branched alkyl group containing 2 to 10 carbon atoms, substituted with a hydroxy group except in the alpha-position, or pharmaceutically acceptable salts thereof.

10. A composition suitable for dermatological application characterised in that it comprises a morpholino compound having the general formula 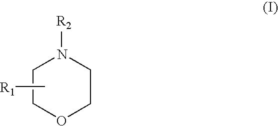
wherein R₁, is a straight or branched alkyl group containing 8 to 16 carbon atoms at the 2- or 3-position of the morpholino ring, and R₂ is a straight or branched alkyl group containing 2 to 10 carbon atoms, substituted with a hydroxy group except in the alpha-position, or pharmaceutically acceptable salts thereof.

11. The composition according to claim 10 wherein the sum of the carbon atoms in the groups R₁ and R₂ is at least 10.

12. The composition according to claim 10, wherein R₂ terminates with a hydroxy group.

13. The composition according to claim 10, wherein the morpholino compound is 3-(4-propyl-heptyl)-4-(2-hydroxyethyl) morpholine.

14. The composition according to claim 10, wherein said composition further comprises an antimicrobial agent.

15. The composition according to claim 14, wherein the antimicrobial agent is an antibiotic selected from the group consisting of clindamycin, erythromycin, benzylpencillin, tetracycline, chloramphenicol, vancomycin and linezolid.

16. The composition according to claim 10, wherein said composition further comprises an anti-inflammatory agent.
